(19) United States
(12) Patent Application Publication
(10) Pub. No.: US 2005/0257310 A1
(43) Pub. Date: Nov. 24, 2005

(54) Golf hat

(76) Inventor: Jong-Hyeon Jang, Seoul (KR)

Correspondence Address: RICHARD M. GOLDBERG, 25 EAST SALEM STREET, SUITE 419, HACKENSACK, NJ, 07601, US

(21) Appl. No.: 10/945,312

(22) Filed: Sep. 20, 2004

(30) Foreign Application Priority Data
May 19, 2004 (KR) P2004-35641
Jun. 25, 2004 (KR) U2004-18104

(51) Int. Cl. A61F 9/00

(52) U.S. Cl. 2/209.13; 2/10

(57) ABSTRACT

A golf hat includes a head covering having a visor extending forwardly therefrom; a substantially U-shaped blind, the blind including opposite foldable sides, each with a first attaching arrangement at a respective end for securing the end of the respective side to a bottom of the visor, an assistant panel attached to each side, a bottom panel which is connected to a front portion of both sides and which has a tab portion with a second attaching arrangement at a center of the bottom panel; and a third attaching arrangement installed in center, left and right sides of the bottom of the visor for securement with the first and second attaching arrangements.

### BACKGROUND OF THE INVENTION

1. Field of the invention

The present invention relates to a golf hat and more particularly, a golf hat comprising a [symbol] shape blind attached to the bottom of the visor via attaching means. Said golf hat is designed to improve concentration in the putting and/or in the approaching shot. If a golfer put on said hat, he can concentrate on the putting line and thereby avoid head up and prevent a shot-miss.

Conventional golf hats have been used as a sun-visor. Recently, a golf hat was designed to prevent head-up when swinging. The Korean Utility Model registration No. 229136 discloses a golf hat which has a blind attached to the one side of the bottom of the visor. However, this hat cannot sufficiently block the side of golfer's eye. Thus, this hat cannot provide the desired concentration in the green.

Also, the Korean laid-open patent publication discloses a golf hat in which cloth blinds are attached to the left and right sides of the bottom of the visor. However, said hat interrupts the concentration because the blinds are shaken when using. The prior application of the present applicant discloses a golf hat in which foldable blinds are attached to the left and right sides of the bottom of the visor. However, it allows eyes to be fixed to the left and right directions only, not to the front and rear directions.

### SUMMARY OF THE INVENTION

The present invention was designed to solve aforementioned problems. An object of the present invention is to provide a golf hat which can make a golfer concentrate on one direction to prevent head-up when putting and/or making an approaching shot.

The golf hat according to the present invention comprises a [symbol] shape blind and an attaching means for binding the blind to the bottom of the visor. Said blind comprises foldable both sides with attaching means at respective end, an assistant panel attached to each side and the bottom panel which is connected to the front portion of both sides and has a knob portion with attaching means at the center. Said attaching means are installed in the center, left and right sides of the bottom of the visor.

When a golfer needs to concentrate in a putting or an approaching shot near the green, he just dispatches the knob portion from the visor. By doing this, the blind of the golf hat of the present invention is fallen down and unfolded. Simultaneously, the bottom panel is also fallen down. Under these circumstances, the golfer can concentrate on one direction and thus avoid head-up and shot miss. When a golfer does not need the blind, he just attaches the knob portion with the attaching means at the center of the bottom of the visor.

### BRIEF DESCRIPTION OF THE DRAWINGS

The other objects and features of the present invention will be hereinafter explained in detail with reference to the accompanying drawings.

FIG. 1 is a resolved perspective view of the present invention.

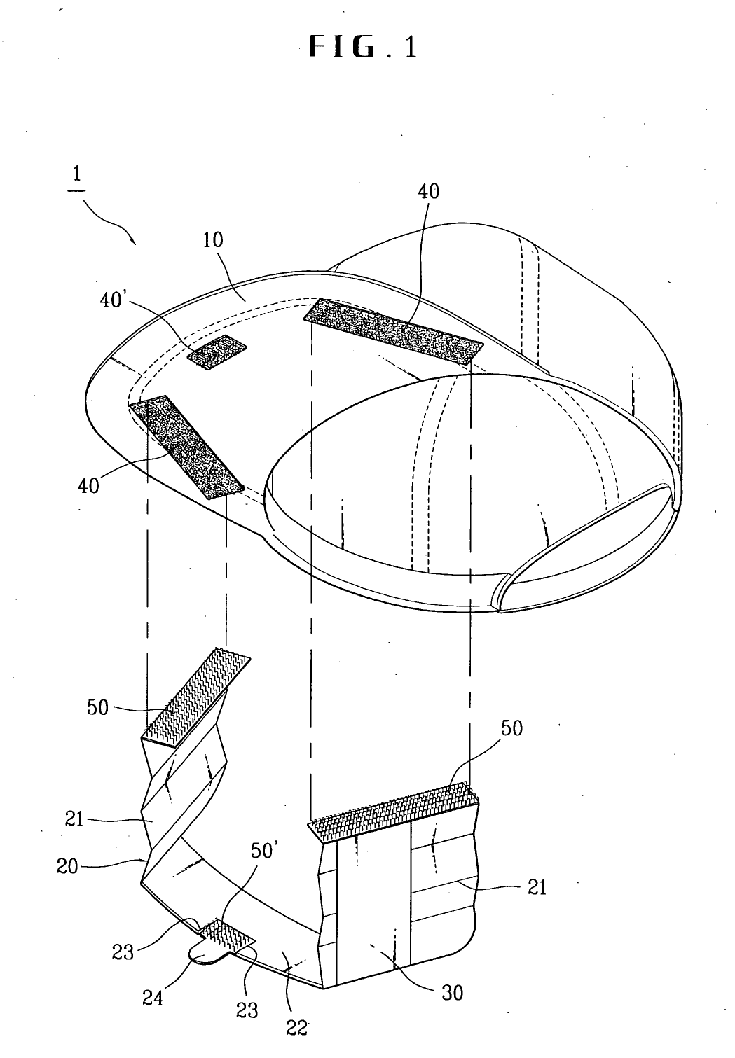

FIG. 2 is a bottom illustrative view of showing a unfolded state of the present invention.

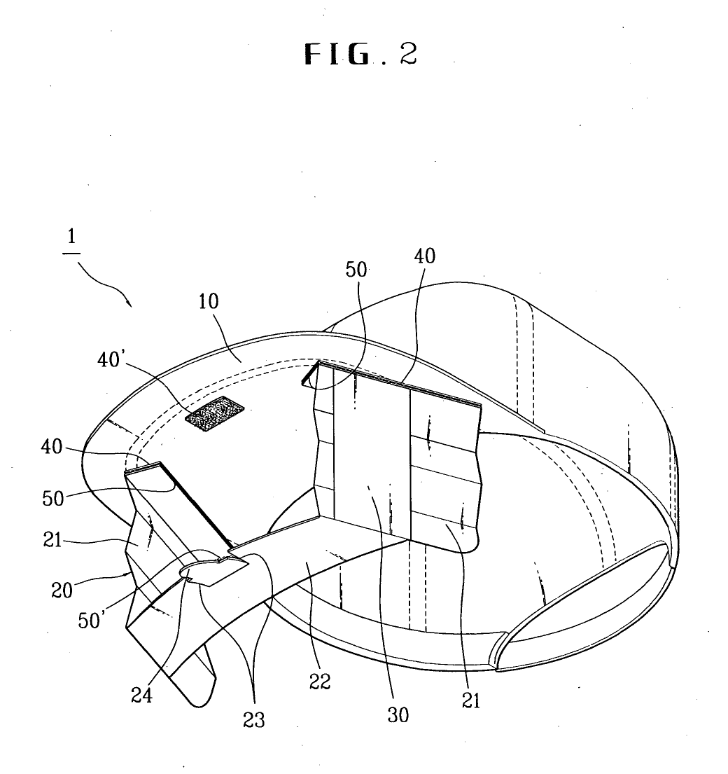

FIG. 3 is a bottom illustrative view of showing a folded state of the present invention.

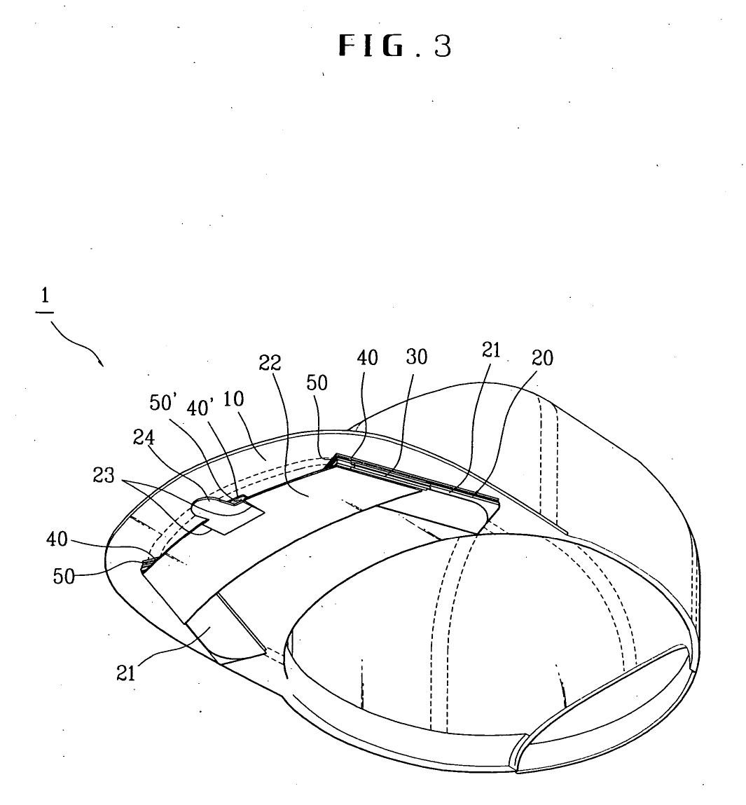

FIG. 4 is a front illustrative view of showing a folded state of the present invention.

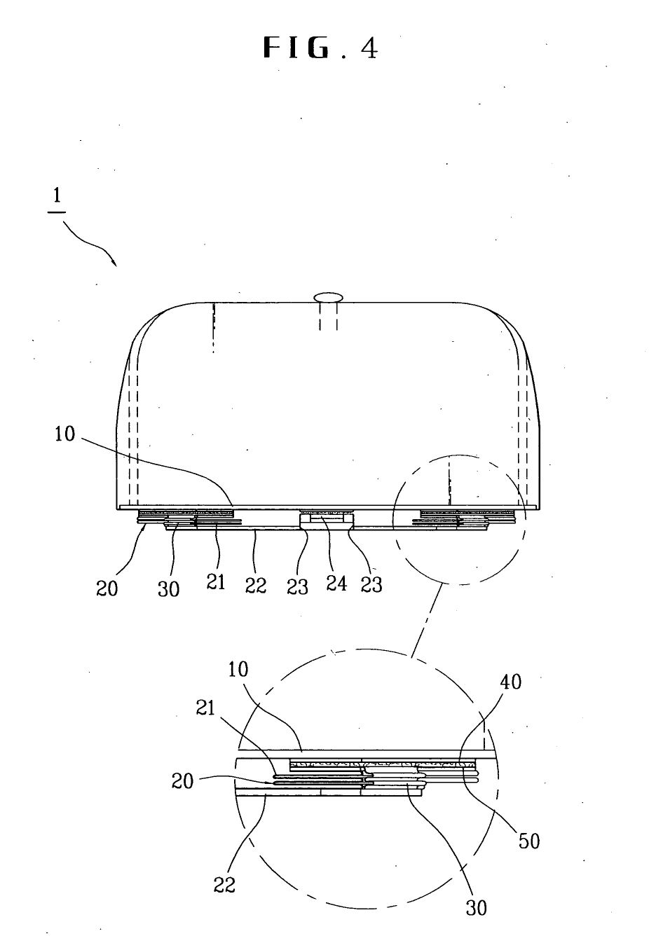

FIG. 5 is a front illustrative view of showing a unfolded state of the present invention.

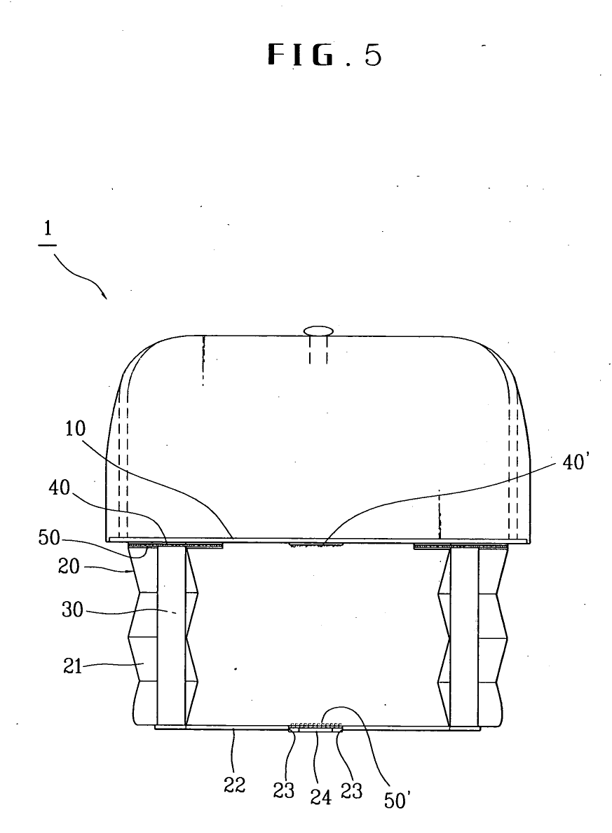

FIG. 6 is a side illustrative view of showing a unfolded state of the present invention.

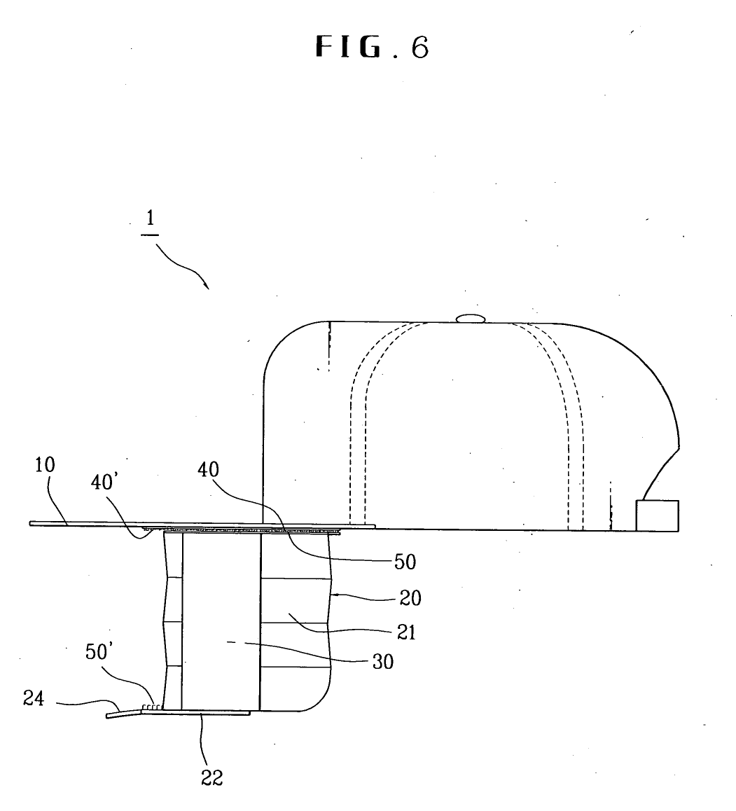

FIG. 7 is an illustrative view of showing a state of using the present invention by a golfer.

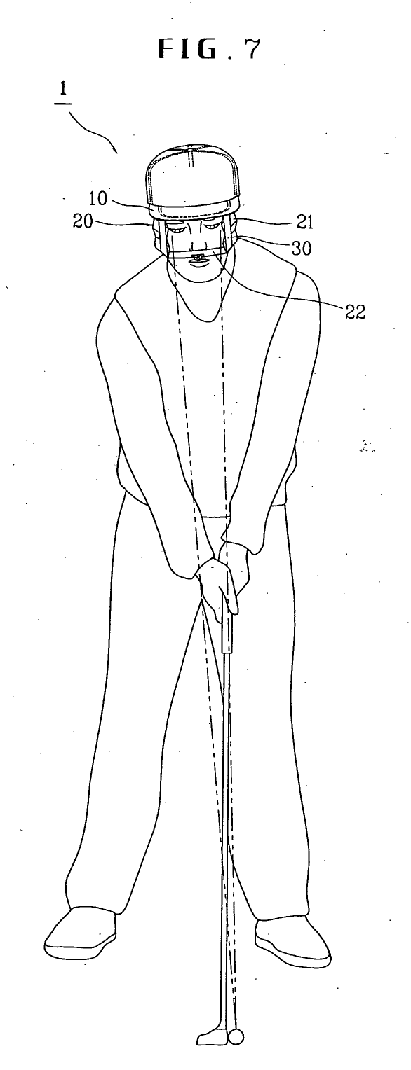

FIG. 8 is a resolved perspective view of other example of the present invention.

FIG. 9 is a bottom illustrative view of showing a combined and unfolded state of the example of FIG. 8.

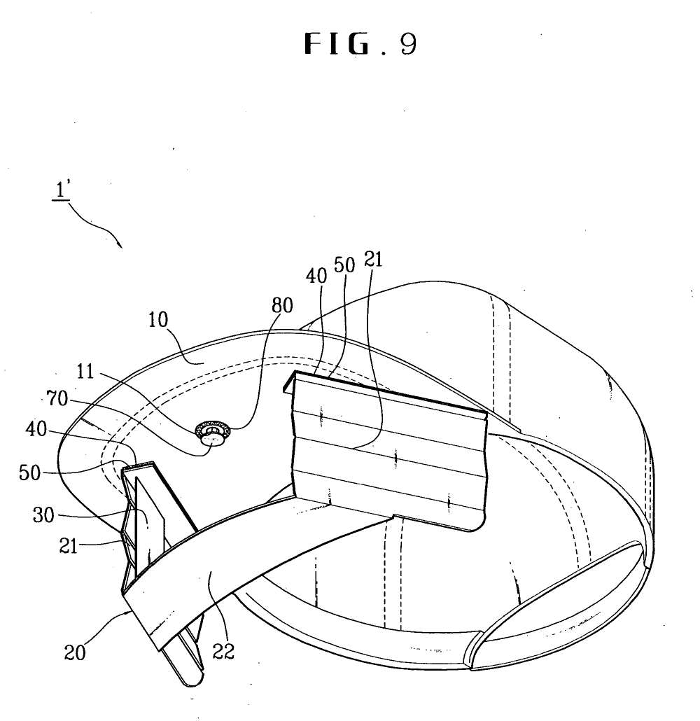

FIG. 10 is a bottom illustrative view of showing a combined and folded state of the example of FIG. 8.

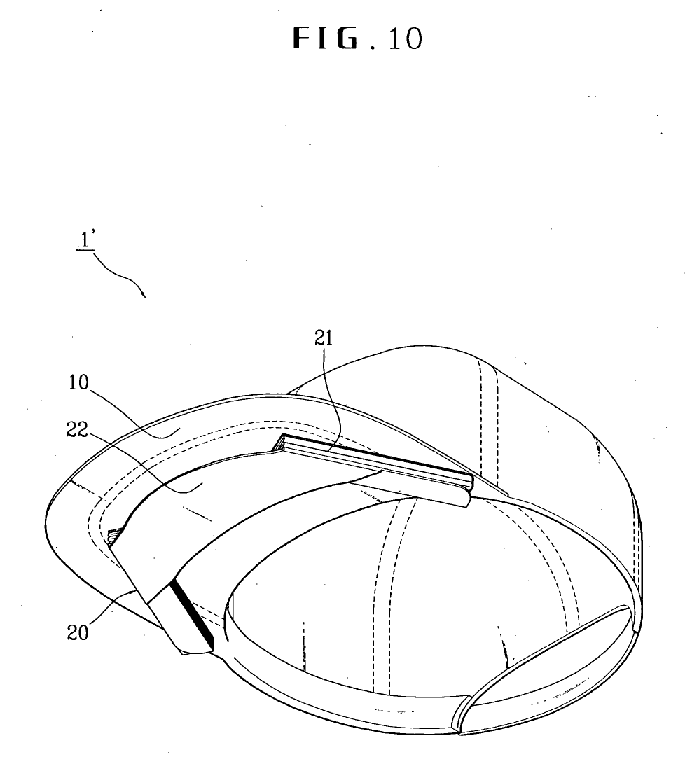

FIG. 11 is a front illustrative view of showing a combined and folded state of the example of FIG. 8.

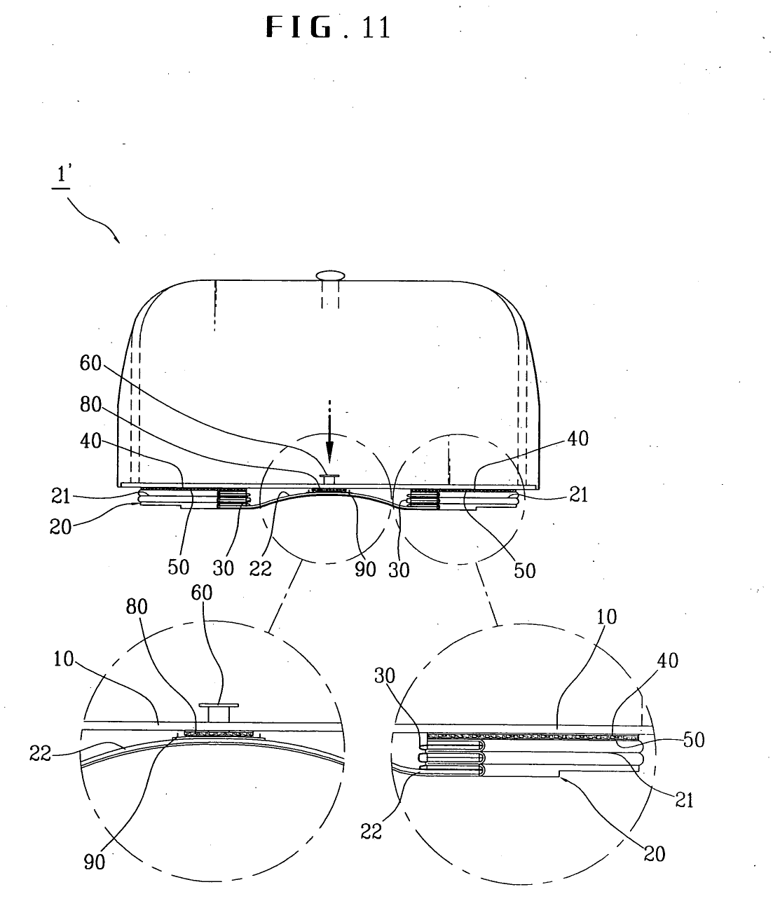

FIG. 12 is a front illustrative view of showing a combined and unfolded state of the example of FIG. 8.

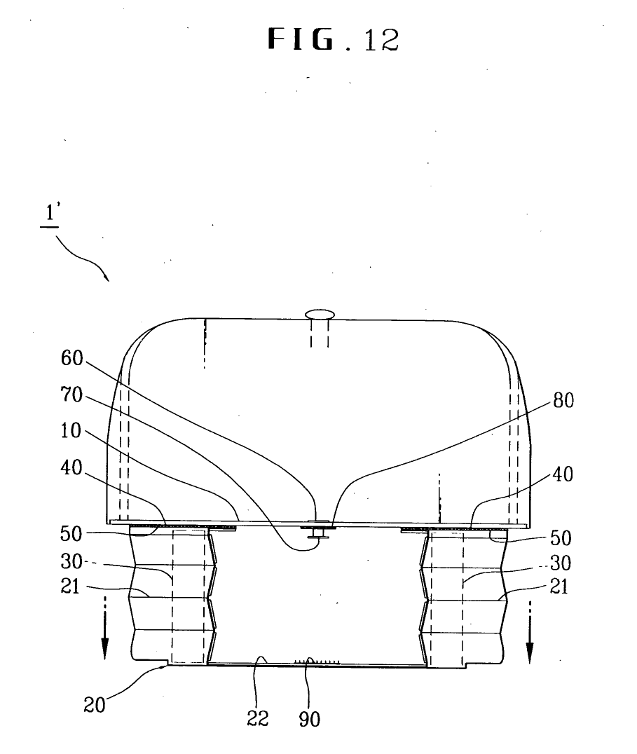

FIG. 13 is a side illustrative view of showing a combined and unfolded state of the example of FIG. 8.

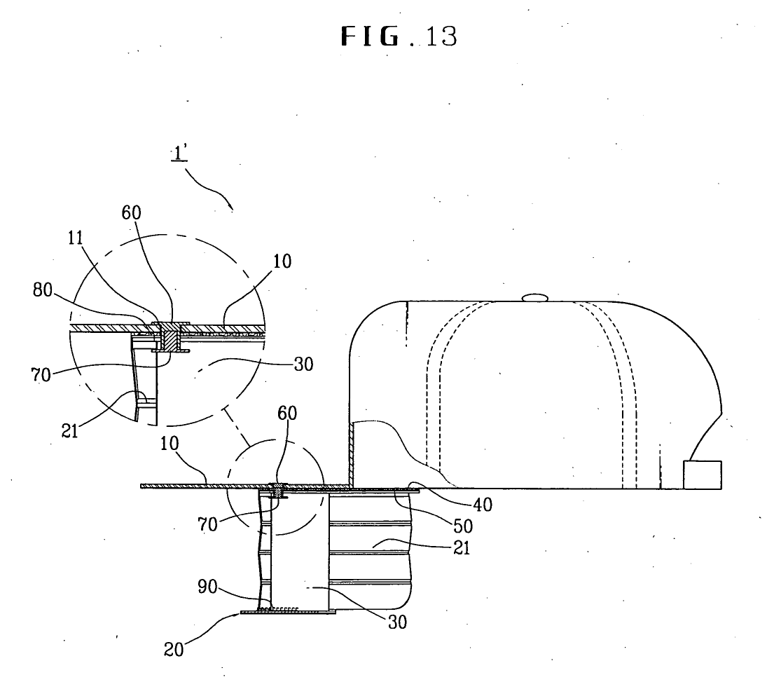

### DETAILED DESCRIPTION OF THE INVENTION

The golf hat 1 according to the present invention comprises a blind 20 and an attaching means 40 for binding the blind on the bottom of the visor 10. Said blind is made in [symbol] shape with attaching means 50 at respective end, which comprises foldable both sides 21, an assistant panel 30 attached to each side and the bottom panel 22 which is connected to the front portion of both sides 21 and has a knob portion 24 with cutting lines 23 at both ends and with attaching means 50′ at the center. Said attaching means are installed in the center, left and right sides of the bottom of the visor. The attaching means 50′ is combined with that 40′ positioned on the bottom center of the visor 10.

The [symbol] shape blind 20 is fixed to the bottom of the visor by attaching means 40, 50. Both foldable sides 21 are folded up and the bottom panel 22 is also attached to the bottom center of the visor 10 via combination of attaching means 50′ and attaching means 40′.

An attaching means includes Velcro but is not limited thereto.

When a golfer tries to make an approaching shot or putting near green, he just pulls down the knob portion 24 of the bottom panel 22, which is combined with the bottom of the visor 10. Then, the knob portion 24 is easily detached from the attaching means 40′ 50′ and the folded sides 21 of the blind 20 are unfolded. Thus, a golfer can naturally concentrate on the inside portion which surrounded by the visor, bottom panel and both sides. Since the golfer's eyes are focused on the one portion, he can avoid head-up and shot miss. The assistant panels 30 which are fixed to each foldable side 21 on several sites control unfolding degree to prevent excessive unfolding. They also help to maintain folding line and make the refolding easily.

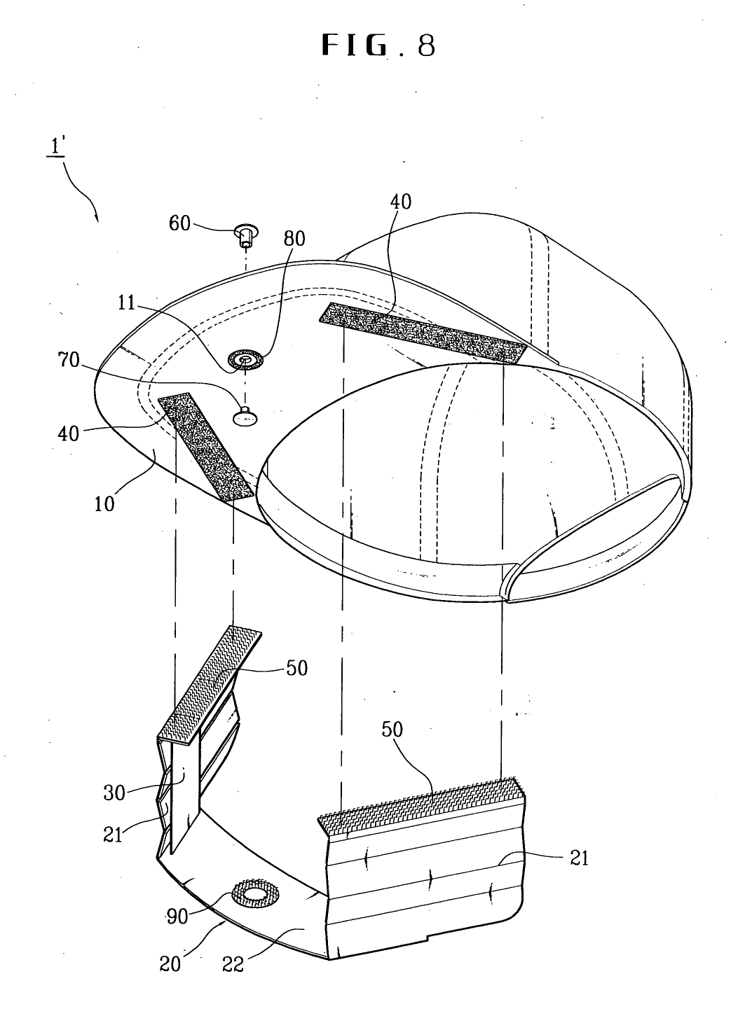

FIG. 8 illustrates another embodiment of the present invention. In said embodiment, a golf hat 1′ comprises a [symbol] shape blind, an attaching means 40 and a hole 11 for inserting push-operation button 60, 70. Said blind comprises foldable both sides 21 with attaching means 50 at respective end, an assistant panel 30 attached to the inside of each side, and the bottom panel 22 which is connected to the front portion of both sides 21 and has an attaching means 90 at its center. Said hole 11 is made at the center of the visor and has an attaching means 80 at its circumference. When a golfer tries to make an approaching shot or putting near green, if he just push the push button 60 inserted in the hole 11, the operation button 70 makes the blind combined with the visor via attaching means 80, 90 dispatch. Then, the folded sides 21 of the blind 20 which were attached with the visor are unfolded. Thus, a golfer can naturally concentrate on the inside portion which surrounded by the visor 10, bottom panel 22 and both sides 21. Since the golfer's eyes are focused on the one portion, he can avoid head-up and shot miss. The assistant panels 30 are fixed to the inside of each foldable side 21 on several sites and thus solve the appearance problem which caused by positioning the assistant panels outside. The assistant panels not only control unfolding degree to prevent excessive unfolding but also help to maintain folding line and make the refolding easily.

As described above, the golf hat 1 of the present invention enables a golfer to concentrate on the putting line. Thus, the golf hat can prevent head-up and shot miss. The golf hat of the present invention is especially useful for the beginner or a golfer who is suffering from low concentration. The blind can be installed even to a golf cap without crown.

1. A golf hat comprising a [symbol] shape blind and an attaching means for combining the blind to the bottom of the visor:
said blind comprising foldable both sides with attaching means at respective end, an assistant panel attached to each side, the bottom panel which is connected to the front portion of both sides and has a knob portion with attaching means at the center; and
said attaching means being installed in the center, left and right sides of the bottom of the visor.

1. A golf hat comprising:
a head covering having a visor extending forwardly therefrom, a substantially U-shaped blinds, said blind comprising:
opposite foldable sides, each with a first attaching arrangement at a respective end for securing the end of the respective side to a bottom of the visor,
an assistant panel attached to each side,
a bottom panel which is connected to a front portion of both sides and which has a tab portion with a second attaching arrangement at a center of the bottom panel; and
a third attaching arrangement installed in center, left and right sides of the bottom of the visor for securement with the first and second attaching arrangements.

2. A golf hat comprising:
a head covering having a visor extending forwardly therefrom,
a substantially U-shaped blinds, said blind comprising.
opposite foldable sides, each with a first attaching arrangement at a respective end for securing the end of the respective side to a bottom of the visor,
an assistant panel attached to an inside of each side,
a bottom panel which is connected to a front portion of both sides and which has a second attaching arrangement at a center of the bottom panel; and
a third attaching arrangement installed in left and right sides of the bottom of the visor for securement with the first attaching arrangements;
a hole at a center of the visor and having a fourth attaching arrangement at a circumference thereof for securement with the second attaching arrangement; and
a push button inserted in the hole for releasing said blind from said visor.
